(19) United States
(12) Patent Application Publication
(10) Pub. No.: US 2025/0067738 A1
(43) Pub. Date: Feb. 27, 2025

(54) STATISTICAL ANALYSIS OF THE OVARIAN TUMOR IMMUNE MICROENVIRONMENT USING MULTIPLEX IMMUNOFLUORESCENCE

(71) Applicant: H. Lee Moffitt Cancer Center and Research Institute, Inc., Tampa, FL (US)

(72) Inventor: Brooke L. Fridley, Prairie Village, KS (US)

(21) Appl. No.: 18/809,650

(22) Filed: Aug. 20, 2024

Related U.S. Application Data
(60) Provisional application No. 63/578,402, filed on Aug. 24, 2023.

(51) Int. Cl. G01N 33/569; G01N 33/574

(52) U.S. Cl. CPC G01N 33/56972; G01N 33/57484; G01N 2333/7051; G01N 2333/70517; G01N 2333/70596

(57) ABSTRACT

The recent development of immunotherapies has ushered in a new era of cancer treatment. These therapeutics have led to revolutionary breakthroughs; however, the efficacy has been modest and is often restricted to a subset of patients. Hence, identification of which cancer patients will benefit from immunotherapy is essential. Multiplex immunofluorescence (mIF) microscopy allows for the assessment and visualization of the tumor immune microenvironment (TIME).

### CROSS-REFERENCE TO RELATED APPLICATIONS

This application claims benefit of U.S. Provisional Application No. 63/578,402, filed Aug. 24, 2023, which is hereby incorporated herein by reference in its entirety.

### STATEMENT OF GOVERNMENT INTEREST

This invention was made with Government Support under Grant Nos. CA218681, CA237318, and CA142081 awarded by the National Institutes of Health. The Government has certain rights in the invention.

### BACKGROUND OF THE INVENTION

The recent development of immunotherapies has ushered in a new era of cancer treatment. These therapeutics have led to revolutionary breakthroughs; however, the efficacy has been modest and is often restricted to a subset of patients. Hence, identification of which cancer patients will benefit from immunotherapy is essential. Multiplex immunofluorescence (mIF) microscopy allows for the assessment and visualization of the tumor immune microenvironment (TIME).

### SUMMARY OF THE INVENTION

Disclosed herein is a method for predicting responsiveness of a subject with high grade serous ovarian cancer to immunotherapy, comprising multiplex immunofluorescence (mIF) microscopy for CD3, CD8, and FoxP3, wherein high abundance but low spatial cluster of CD3+ cells and CD3+CD8+ cells indicates better overall survival and responsiveness to immunotherapy. In some embodiments, high abundance but low spatial cluster of CD3+ cells and CD3+CD8+ cells is detected, and the method further involves treating the subject with immunotherapy.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A and 1B show tissue microarray core of a high grade serous ovarian cancer tumor illuminated by fluorescence bound to markers of interest (FIG. 1A) and plotted using the spatial locations of identified cells and markers (FIG. 1B).

FIGS. 2A to 2D show TMA cores displaying examples of high abundance/high degree of spatial clustering (FIG. 2A), high abundance/low degree of spatial clustering (FIG. 2B), low abundance/high degree of spatial clustering (FIG. 2C), and low abundance/low degree of spatial clustering (FIG. 2D) of cytotoxic T cells in high grade serous ovarian cancer tumor compartments.

FIGS. 3A to 3F show univariate clustering survival curves where TMA cores are categorized based on their phenotype abundance (Absent/High/Low, or Absent/Present) and degree of spatial clustering (High/Low) into 5 levels (HH, HL, LH, LL, and Absent) or 3 levels (PH, PL, and Absent).

FIGS. 4A to 4F show univariate clustering survival curves after cores were subset to either high abundance in markers with 5 abundance/spatial levels or present cells in markers with 3 levels to show survival difference in degree of spatial clustering in the higher abundance samples.

FIGS. 5A to 5E show bivariate clustering survival curves where TMA cores are categorized by two cell phenotype abundances (Absent or Present of the first and second marker combination, respectively) and degree of spatial colocalization (None, High, or Low).

FIGS. 6A to 6E show bivariate clustering survival curves restricting to cores that had both marker combinations present to determine overall survival difference between high and low degree of spatial clustering.

FIGS. 7A to 7J show bivariate clustering. Survival curves where TMA cores were categorized by their two phenotype abundances (Absent or Present) and degree of spatial clustering/colocalization (None, High, or Low) for all core samples (FIGS. 7A, 7C, 7E, 7G, 7I) and subset to only cores with both phenotypes present (PPH or PPL) (FIGS. 7B, 7D, 7F, 7H, and 7J). Analysis treated CD3+CD8+ or CD3+CD8+CD69+ phenotype as marker combination around either Treg or helper T cells (i.e., the complement of the analysis presented in FIG. 2). APN=absence of CD3+CD8+ (A), presence of other phenotypes (CD3+CD4+, CD3+CD4+Foxp3+, or CD3+CD4+CD69+) (P), and no spatial clustering computed (N); PAN=presence of CD3+CD8+ or CD3+CD8+CD69+ (P), absence of other phenotypes (CD3+CD4+, CD3+CD4+Foxp3+, or CD3+CD4+CD69+) (A), and no spatial clustering computed (N); PPH=presence of both phenotypes and high level of colocalization; PPL=presence of both phenotypes and low level of colocalization; AAN=absence of both phenotypes and no level of colocalization computed.

FIG. 8 contains Supplementary Table 1 providing markers used for the detection of cell phenotypes with the Vectra®3 Automated Quantitative Pathology Imaging System.

FIG. 9 contains Supplementary Table 2 providing T cell phenotypes considered in clustering and colocalization analyses and the percentage of cores (n=1244) that had at least one cell positive for that marker combination and at least two cells positive for the marker combination allowing the degree of spatial clustering to be computed.

FIG. 10 contains Supplemental Table 3 providing Weighted Kappa for core assignments (HH, HL, LH, LL, and Absent) comparing the different marker combinations for the 1244 TMA cores. The marker for recently activated helper T cells has been removed below because it produces only three levels (absent, present high, and present low).

FIG. 11 contains Supplementary Table 4 providing Bivariate Clustering. Survival analysis results from colocalization of CD3+CD4+Foxp3+, CD3+CD4+, or CD3+CD4+CD69+ cells with CD3+CD8+ or CD3+CD8+CD69+ using bivariate Ripley's K (median as threshold for low (L) vs high (H)) with cell abundance (present (P) vs absent (A)). Models were adjusted for age at diagnosis, high/low stage, and patient cohort and CD3 abundance for T cell subsets. APN=absent of CD3+CD4+, CD3+CD4+Foxp3+, or CD3+CD4+CD69+ (A), present of other phenotype (CD3+CD8+ or CD3+CD8+CD69+) (P), and no spatial clustering computed (N); PAN=presence of CD3+CD4+, CD3+CD4+Foxp3+, or CD3+CD4+CD69+ (P), absence of other phenotype (CD3+CD8+ or CD3+CD8+CD69+) (A), and no spatial clustering computed (N); PPH=presence of both phenotypes and high level of colocalization; PPL=presence of both phenotypes and low level of colocalization; AAN=absence of both phenotypes and no level of colocalization computed. Bolded values indicate results that are statistically significant with p-value<0.01.

FIG. 12 contains Supplementary Table 5 providing Univariate Clustering of White (non-Hispanic). Survival analysis results using cellular abundance (1% threshold for low vs high) and Ripley's K degree of clustering (median as threshold for low vs high). Models were adjusted for age at diagnosis, high/low stage, and patient cohort and CD3 abundance for T cell subsets. Absent=no immune marker present in tumor core; LL=low abundance/low spatial clustering, LH=low abundance/high spatial clustering; HL=high abundance/low spatial clustering; HH=high abundance/high spatial clustering. Bolded values indicate results that are statistically significant.

FIG. 13 contains Supplementary Table 6 providing Univariate Clustering of Patients in the Last 30 Years. Survival analysis results using cellular abundance (1% threshold for low vs high) and Ripley's K degree of clustering (median as threshold for low vs high). Models were adjusted for age at diagnosis, high/low stage, and patient cohort and CD3 abundance for T cell subsets. Absent=no immune marker present in tumor core; LL=low abundance/low spatial clustering, LH=low abundance/high spatial clustering; HL=high abundance/low spatial clustering; HH=high abundance/high spatial clustering. Bolded values indicate results that are statistically significant.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “spatial clustering” refers to a non-random spatial distribution of cells.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. The specificity of CAR designs may be derived from ligands of receptors (e.g., peptides). In some embodiments, a CAR can target cancers by redirecting the specificity of a T cell expressing the CAR specific for tumor associated antigens.

The term “T cell” refers to T lymphocytes as defined in the art and is intended to include thymocytes, immature T lymphocytes, mature T lymphocytes, resting T lymphocytes, or activated T lymphocytes. The T cells can be CD4+ T cells, CD8+ T cells, CD4+CD8+ T cells, or CD4−CD8− cells. The T cells can also be T helper cells, such as T helper 1 (TH1), or T helper 2 (TH2) cells, or TH17 cells, as well as cytotoxic T cells, regulatory T cells, natural killer T cells, naïve T cells, memory T cells, or gamma delta T cells.

The T cells can be a purified population of T cells, or alternatively the T cells can be in a population with cells of a different type, such as B cells and/or other peripheral blood cells. The T cells can be a purified population of a subset of T cells, such as CD4+ T cells, or they can be a population of T cells comprising different subsets of T cells. In another embodiment of the invention, the T cells are T cell clones that have been maintained in culture for extended periods of time. T cell clones can be transformed to different degrees. In a specific embodiment, the T cells are a T cell clone that proliferates indefinitely in culture.

In some embodiments, the T cells are primary T cells. The term “primary T cells” is intended to include T cells obtained from an individual, as opposed to T cells that have been maintained in culture for extended periods of time. Thus, primary T cells are particularly peripheral blood T cells obtained from a subject. A population of primary T cells can be composed of mostly one subset of T cells. Alternatively, the population of primary T cells can be composed of different subsets of T cells.

### Multiplex Immunofluorescence (MIF) Microscopy

In some embodiments of the present invention, an image processing tool (e.g., image processing tool) is provided that generates digitized images of tumor biopsies subject to immunofluorescence (IF) (e.g., multiplex IF) CD3, CD4, CD8, and CD69. In multiplex immunofluorescence (mIF) microscopy, multiple proteins in a tissue specimen are simultaneously labeled with different fluorescent dyes conjugated to antibodies specific for each particular protein. Each dye has a distinct emission spectrum and binds to its target protein on immune cells.

Cell types represented by immune markers include stem cells (CD34⁺CD31⁻CD117⁺), granulocytes (CD45⁺CD11b⁺CD15⁺CD24⁺CD114⁺CD182⁺), monocytes (CD4⁺CD45⁺CD14⁺CD114⁺CD11a⁺CD11b⁺CD91⁺CD16⁺), T lymphocytes (CD45⁺CD3⁺), T helper cells (CD45⁺CD3⁺CD4⁺), T regulatory cells (CD4⁺CD25⁺FoxP3⁺), Cytotoxic T cells (CD45⁺CD3⁺CD8⁺), B lymphocytes (CD45⁺CD19⁺CD20⁺CD24⁺CD38⁺CD22⁺), thrombocytes (CD45⁺CD61⁺), and natural killer cells (CD16⁺CD56⁺CD3 CD31⁺CD30⁺CD38⁺). In addition, CD69 is a marker for activation, and TIM3 and LAG3 are markers for exhaustion.

In some embodiments, multiplex immunofluorescence (mIF) staining can be combined with quantitative digital image analysis for the characterization of the tumor immune microenvironment (TIME). Generally, mIF data is used to examine the abundance of immune cells in the TIME; however, this does not capture spatial patterns of immune cells throughout the TIME, a metric increasingly recognized as important for prognosis. To address this gap, an R package spatialTIME was developed that enables spatial analysis of mIF data, as well as the iTIME web application that provides a robust but simplified user interface for describing both abundance and spatial architecture of the TIME. These are described in Creed et al. Bioinformatics. 2021 37 (23): 4584-4586, which is incorporated by reference for these methods.

The spatialTIME package calculates univariate and bivariate spatial statistics (e.g. Ripley's K, Besag's L, Macron's M and G or nearest neighbor distance) and creates publication quality plots for spatial organization of the cells in each tissue sample. The iTIME web application allows users to statistically compare the abundance measures with patient clinical features along with visualization of the TIME for one tissue sample at a time.

The degree of spatial clustering for each immune cell type can be computed based on the empirical distribution of the Ripley's K statistics under CSR (e.g., observed Ripley's K minus the mean of the empirical distribution of Ripley's K under CSR) (Wilson, et al. PLOS Computational Biology, 2022 18 (3): e1009900). The degree of spatial clustering can be defined as high (>median) or low (≤median). The cell abundance can also be defined as absent, low (≤1% of total cells), or high (>1% of total cells). This results in a 5 level categorical variable for the statistical analysis: absent, low abundance/low spatial clustering (LL), low abundance/high spatial clustering (LH), high abundance/low spatial clustering (HL), and high abundance/high spatial clustering (HH).

As disclosed herein, high abundance and low spatial cluster (HL) of CD3+CD4⁻CD8⁻ cells (TILs), and high or low abundance and low spatial cluster (HL or LL) of CD3+CD4⁻CD8⁺CD69⁻ cells (CTLs) indicates better overall survival and responsiveness to immunotherapy.

### Cancers

The cancer treated by the disclosed compositions and methods can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, and mesentery cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colorectal cancer, and hepatobiliary cancer.

In some embodiments the cancer is a solid tumor. In some embodiments the cancer is melanoma, ovarian, breast, or colorectal cancer. In some embodiments, the cancer is a high grade serous ovarian cancer.

### Immunotherapy

In some embodiments, the immunotherapy is a chimeric antigen receptor (CAR) T cell containing CAR polypeptides. A CAR polypeptide is generally made up of three domains: an ectodomain, a transmembrane domain, and an endodomain. The ectodomain is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR). CAR polypeptides generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

Additional CAR constructs are described, for example, in Fresnak A D, et al. Engineered T cells: the promise and challenges of cancer immunotherapy. Nat Rev Cancer. 2016 Aug. 23; 16 (9): 566-81, which is incorporated by reference in its entirety for the teaching of these CAR models.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD34) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD34 chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4 (2): 89-99 (2004)). The hinge sequence may be positioned between the antigen recognition moiety (e.g., scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen recognition domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

CAR-T cells involve immune effector cells that are engineered to express CAR polypeptides. These cells are preferably obtained from the subject to be treated (i.e. are autologous). However, in some embodiments, immune effector cell lines or donor effector cells (allogeneic) are used. In still other embodiments, the immune effect cells are not HLA-matched. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some aspects, the disclosed methods involve treating the subject with Adoptive Cell Transfer (ACT) of lymphocytes, such as tumor-infiltrating lymphocytes (TILs), such as HLA-matched TILs.

Tumor-infiltrating lymphocyte (TIL) production is a 2-step process: 1) the pre-REP (Rapid Expansion) stage where you the grow the cells in standard lab media such as RPMI and treat the TILs w/reagents such as irradiated feeder cells, and anti-CD3 antibodies to achieve the desired effect; and 2) the REP stage where you expand the TILs in a large enough culture amount for treating the patients. The REP stage requires cGMP grade reagents and 30-40 L of culture medium. However, the pre-REP stage can utilize lab grade reagents (under the assumption that the lab grade reagents get diluted out during the REP stage), making it easier to incorporate alternative strategies for improving TIL production. Therefore, in some embodiments, the disclosed TLR agonist and/or peptide or peptidomimetics can be included in the culture medium during the pre-REP stage.

Adoptive cell transfer (ACT) is a very effective form of immunotherapy and involves the transfer of immune cells with antitumor activity into cancer patients. ACT is a treatment approach that involves the identification, in vitro, of lymphocytes with antitumor activity, the in vitro expansion of these cells to large numbers and their infusion into the cancer-bearing host. Lymphocytes used for adoptive transfer can be derived from the stroma of resected tumors (tumor infiltrating lymphocytes or TILS). They can also be derived or from blood if they are genetically engineered to express antitumor T cell receptors (TCRs) or chimeric antigen receptors (CARs), enriched with mixed lymphocyte tumor cell cultures (MLTCs), or cloned using autologous antigen presenting cells and tumor derived peptides. ACT in which the lymphocytes originate from the cancer-bearing host to be infused is termed autologous ACT. US 2011/0052530 relates to a method for performing adoptive cell therapy to promote cancer regression, primarily for treatment of patients suffering from metastatic melanoma, which is incorporated by reference in its entirety for these methods.

ACT may be performed by (i) obtaining autologous lymphocytes from a mammal, (ii) culturing the autologous lymphocytes to produce expanded lymphocytes, and (ii) administering the expanded lymphocytes to the mammal. Preferably, the lymphocytes are tumor-derived, i.e. they are TILs, and are isolated from the mammal to be treated, i.e. autologous transfer.

Autologous ACT as described herein may also be performed by (i) culturing autologous lymphocytes to produce expanded lymphocytes; (ii) administering nonmyeloablative lymphodepleting chemotherapy to the mammal; and (iii) after administering nonmyeloablative lymphodepleting chemotherapy, administering the expanded lymphocytes to the mammal.

Autologous TILs may be obtained from the stroma of resected tumors. Tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase).

Expansion of lymphocytes, including tumor-infiltrating lymphocytes, such as T cells can be accomplished by any of a number of methods as are known in the art. For example, T cells can be rapidly expanded using non-specific T-cell receptor stimulation in the presence of feeder lymphocytes and interleukin-2 (IL-2), IL-7, IL-15, IL-21, or combinations thereof. The non-specific T-cell receptor stimulus can e.g. include around 30 ng/ml of OKT3, a mouse monoclonal anti-CD3 antibody (available from Ortho-McNeil®, Raritan, N.J. or Miltenyi Biotec, Bergisch Gladbach, Germany). Alternatively, T cells can be rapidly expanded by stimulation of peripheral blood mononuclear cells (PBMC) in vitro with one or more antigens (including antigenic portions thereof, such as epitope(s), or a cell of the cancer, which can be optionally expressed from a vector, such as an human leukocyte antigen A2 (HLA-A2) binding peptide, e.g., approximately 0.3 UM MART-1:26-35 (27 L) or gp100: 209-217 (210M)), in the presence of a T-cell growth factor, such as around 200-400 III/ml, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

In some embodiments, nonmyeloablative lymphodepleting chemotherapy is administered to the mammal prior to administering to the mammal the expanded tumor-infiltrating lymphocytes. The purpose of lymphodepletion is to make room for the infused lymphocytes, in particular by eliminating regulatory T cells and other non-specific T cells which compete for homeostatic cytokines Nonmyeloablative lymphodepleting chemotherapy can be any suitable such therapy, which can be administered by any suitable route known to a person of skill. The nonmyeloablative lymphodepleting chemotherapy can comprise, for example, the administration of cyclophosphamide and fludarabine, particularly if the cancer is melanoma, which can be metastatic. A preferred route of administering cyclophosphamide and fludarabine is intravenously. Likewise, any suitable dose of cyclophosphamide and fludarabine can be administered. Preferably, around 40-80 mg/kg, such as around 60 mg/kg of cyclophosphamide is administered for approximately two days after which around 15-35 mg/m2, such as around 25 mg/m2 fludarabine is administered for around five days, particularly if the cancer is melanoma.

Specific tumor reactivity of the expanded TILs can be tested by any method known in the art, e.g., by measuring cytokine release (e.g., interferon-gamma) following co-culture with tumor cells. In one embodiment, the autologous ACT method comprises enriching cultured TILs for CD8+ T cells prior to rapid expansion of the cells. Following culture of the TILs in IL-2, the T cells are depleted of CD4+ cells and enriched for CD8+ cells using, for example, a CD8 microbead separation (e.g., using a CliniMACS<plus>CD8 microbead system (Miltenyi Biotec)). In an embodiment of the method, a T-cell growth factor that promotes the growth and activation of the autologous T cells is administered to the mammal either concomitantly with the autologous T cells or subsequently to the autologous T cells. The T-cell growth factor can be any suitable growth factor that promotes the growth and activation of the autologous T-cells. Examples of suitable T-cell growth factors include interleukin (IL)-2, IL-7, IL-15, IL-12 and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

Preferably, expanded lymphocytes produced by these methods are administered as an intra-arterial or intravenous infusion, which preferably lasts about 30 to about 60 minutes. Other examples of routes of administration include intraperitoneal, intrathecal and intralymphatic. Likewise, any suitable dose of lymphocytes can be administered. In one embodiment, about 1×10¹⁰ lymphocytes to about 15×10¹⁰ lymphocytes are administered.

The disclosed methods can involve treating the subject with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

### Combinations

The disclosed methods can involve treating the subject with a combination of additional therapeutic agents. In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the subject further receives radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

A number of embodiments of the invention have been described. Nevertheless, it will be understood that various modifications may be made without departing from the spirit and scope of the invention. Accordingly, other embodiments are within the scope of the following claims.

### Examples

### Example 1: Spatial Clustering of Cytotoxic and Tumor Infiltrating Lymphocytes is Associated with Overall Survival in Women with High Grade Serous Ovarian Cancer

Ovarian cancer is the fifth leading cause of cancer-related death among women in the United States making it the deadliest gynecologic malignancy (Siegel, et al. Cancer statistics, 2020. CA Cancer J Clin, 2020 70 (1): 7-30). The most common epithelial ovarian cancer (EOC) which accounts for 75% of deaths is high grade serious ovarian cancer (HGSOC), which is most often diagnosed at late stages due to non-specific symptoms (Stewart, et al. Seminars in Oncology Nursing, 2019 35 (2): 151-156; Jayson, et al. Ovarian cancer. The Lancet, 2014 384 (9951): 1376-1388; Kurman, et al. Am J Pathol, 2016 186 (4): 733-47). Recent notable treatment advancements include the use of poly (ADP-ribose) polymerase (PARP) inhibitors and anti-VEGF therapies; however, overall survival (OS) has not substantially changed as these therapeutics improve outcomes for only a subset of patients (Konecny, et al. Br J Cancer, 2016 115 (10): p. 1157-1173. (doi: 10.1038/bjc.2016.311); Liu, et al. Gynecol Oncol, 2014 133 (2): 362-9; Lheureux, et al. C A Cancer J Clin, 2019 69 (4): 280-304; Shaw, et al. Curr Opin Oncol, 2013 25 (5): 558-65). Clinical trials have further employed immune checkpoint inhibitors such as pembrolizumab for PD-1/PD-L1, though, overall response rates have remained low (Borella, et al. Diagnostics (Basel), 2020 10 (3)). Because of this, understanding the spatial contexture of the tumor immune microenvironment (TIME) in HGSOC tumors is important for developing new treatments and identifying individuals who may respond to immunotherapy.

Patients with advanced ovarian carcinoma whose tumors have tumor infiltrating lymphocytes (TILs) have significantly longer progression free survival (PFS) and improved overall survival (OS) compared to those without TILs (Zhang, et al. New England Journal of Medicine, 2003 348 (3): 203-213). Additionally, TIL subtypes in the tumor compartment, such as cytotoxic T lymphocytes (CTLs; CD3+CD8+), which are associated with anti-tumor responses, have been associated with better OS (Sato, et al. Proc Natl Acad Sci USA, 2005 102 (51): 18538-18543). B cells (CD20+) are also related to ovarian cancer-specific survival, especially when found near CTLs, suggesting the importance of the TIME spatial contexture (Milne, et al. PloS one, 2009. 4 (7): e6412-e6412).

Assessing the spatial distribution of immune cells, in addition to overall abundance in the TIME, may produce new insights into novel aspects of the tumor immune response and its role in cancer prognosis (Bao, et al. Am J Public Health, 2016. 106 (9): 1573-81). Hence, to quantify the degree to which specific immune cell types are clustered together or randomly distributed across the TIME, a permutation-based Ripley's K clustering approach was developed to assess the degree of spatial clustering in a univariate (i.e., single cell type clustering) and bivariate (i.e., two cell types colocalization) manner (Wilson, et al. PLOS Computational Biology, 2022 18 (3): e1009900; Creed, et al. Bioinformatics, 2021. 37 (23): 4584-6). In this research, three large epidemiologic studies of ovarian cancer, the Nurses' Health Study (NHS), the Nurses' Health Study II (NHSII), and the New England Case-Control Study (NECC), were leverage to characterize the spatial architecture of T cell subsets in the TIME in HGSOC tumors and assess its association with overall survival.

### Methods

### Study Cohorts

Three large epidemiologic studies were leveraged: Nurses' Health Study (NHS), Nurses' Health Study II (NHSII), and the New England case-control study of ovarian cancer (NECC). The NHS is a prospective cohort study that started in 1976 and enrolled 121,700 female registered nurses aged 30 to 55 years living in the 11 states with the largest number of registered nurses at the time (Bao, et al. Am J Public Health, 2016 106 (9): 1573-81). NHSII began in 1989 with 116,429 women aged 25 to 42 years residing in 14 states. Participants in the NHS and NHSII with incident ovarian cancer diagnoses were identified via self-report on biennial questionnaires and deaths were identified via linkage to the National Death Index (Stampfer, et al. Am J Epidemiol, 1984. 119 (5): 837-9; Rich-Edwards, et al. Am J Epidemiol, 1994 140 (11): 1016-9). The NECC is a population-based ovarian cancer case-control study that enrolled 2,040 women with newly diagnosed epithelial ovarian cancer aged 18 to 80 years residing in Eastern Massachusetts and New Hampshire from 1992 to 2008 (Terry, et al. Cancer Res, 2005 65 (13): 5974-81; Vitonis, et al. Obstet Gynecol, 2011 117 (5): 1042-1050). In all three studies, a gynecologic pathologist reviewed surgical and pathology reports in addition to tumor slides to confirm case diagnoses and ascertained stage, grade, morphology, histology, and circle regions consisting of tumor. The NHS and NHSII study protocols were approved by the Institutional Review Boards of the Brigham and Women's Hospital and Harvard T. H. Chan School of Public Health, and those of participating registries as required. The NECC protocol was approved by the institutional review board of the Brigham and Women's Hospital and Moffitt Cancer Center.

### Measurement of T Cell Markers

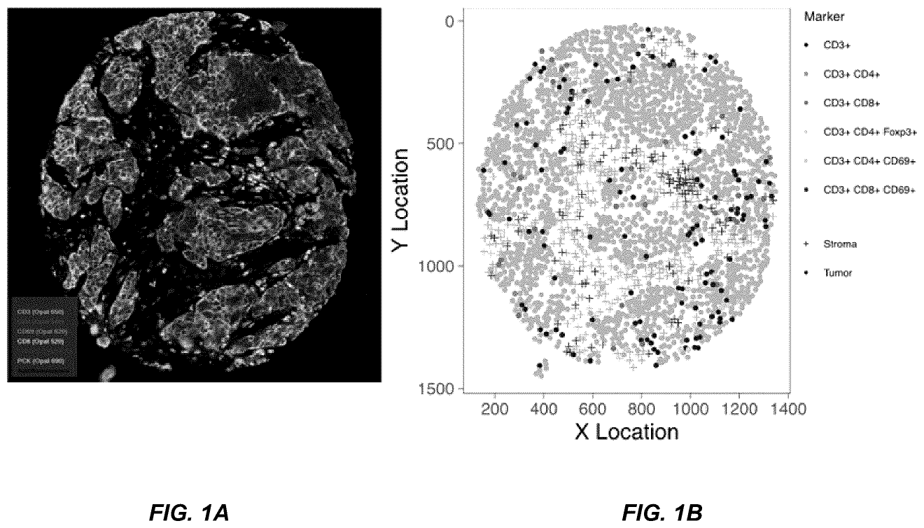

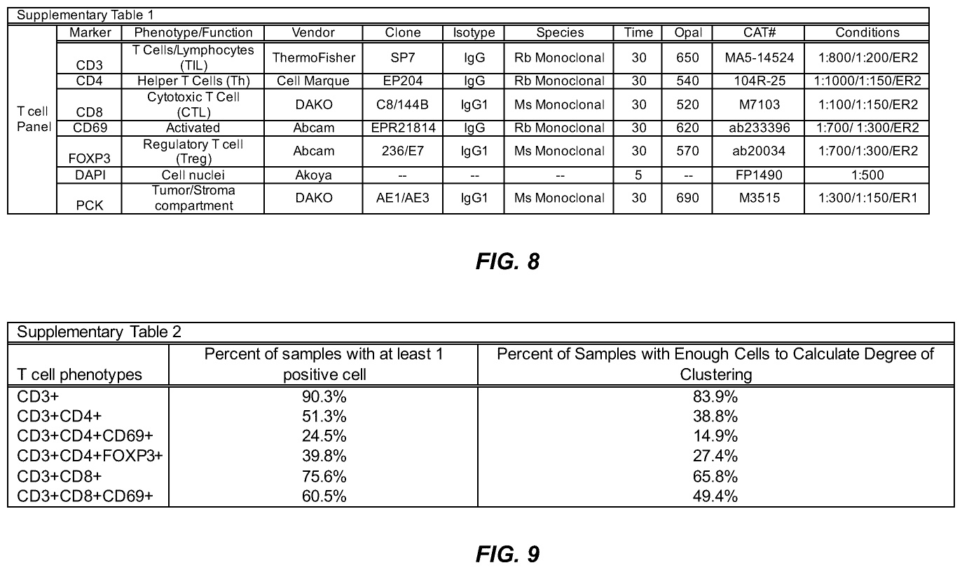

Collection of tumor tissue and measurement of immune markers have been previously described by Hathaway et. al. (2022) (Hathaway, et al. Cancer Epidemiol Biomarkers Prev. 2023 32 (1): 66-73). Briefly, formalin-fixed and paraffin-embedded (FFPE) ovarian tumor tissue samples were collected when information on the surgery was available. These were used to create tissue microarrays (TMAs) with three to six 0.6 mm or 1 mm cores per case. TMAs were immunostained using the AKOYA Biosciences OPAL™ 7-Color Automation IHC Kit (Waltham, MA, USA). Each panel allowed for five antibodies plus DAPI for nuclei detection and pancytokeratin followed by labelling using a tyramide signal amplification-based kit. Following staining, image collection was performed by Vectra®3 Automated Quantitative Pathology Imaging System (0.499 μm/pixel). InForm (AKOYA) was used for spectral unmixing, and HALO (Indica Labs) was used for cell phenotype assignments. For this study, a T-cell panel was used with the following markers: CD3, CD4, CD8, CD69, and Foxp3 (FIG. 8—Supplementary Table 1), with an example of the stained image for a TMA core presented in FIG. 1.

### Spatial Metrics

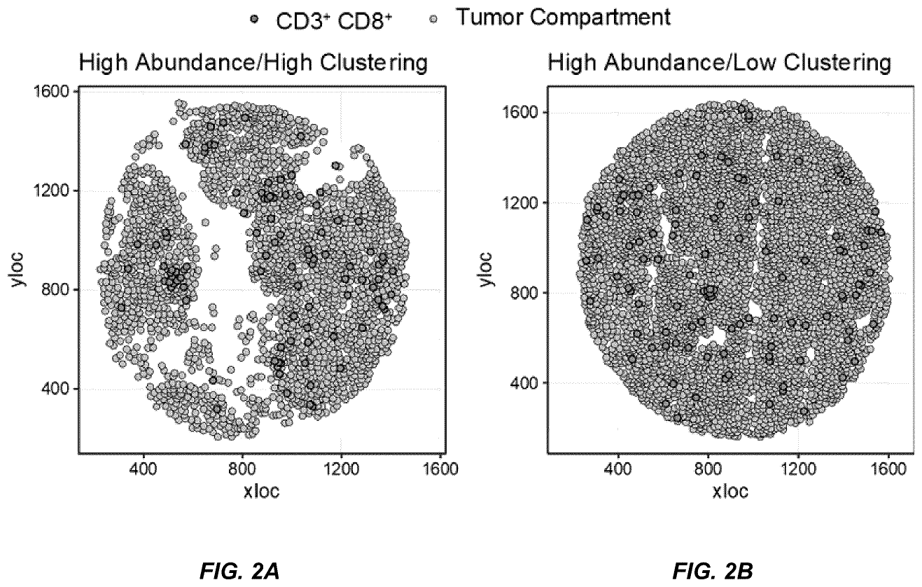

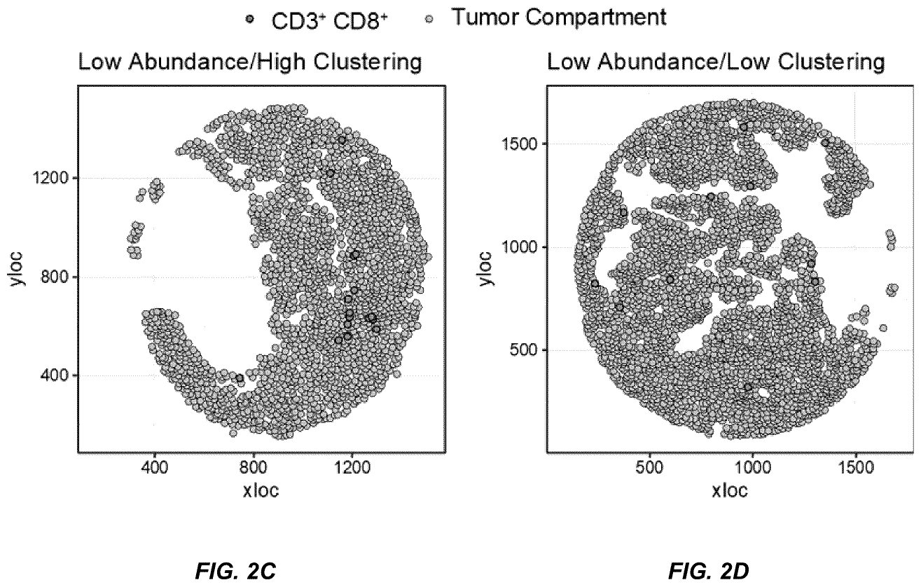

The level of spatial clustering was computed using R v4.2.0 and the spatialTIME package v1.2.1 (Creed, et al. Bioinformatics, 2021 37 (23): 4584-6). Ripley's K was calculated for both univariate (clustering with same cell phenotype) and bivariate (colocalization of two cell phenotypes) at a radius of r=150 pixels (˜75 μm) within the tumor compartment. To address the issue that some TMA cores have large holes with no measured cells a permutation approach was implemented as described (Wilson, et al. PLOS Computational Biology, 2022 18 (3): e 1009900) to assess clustering under complete spatial randomness (CSR; estimated with 100 permutations). The degree of spatial clustering was computed based on the empirical distribution of the Ripley's K statistics under CSR (e.g., observed Ripley's K minus the mean of the empirical distribution of Ripley's K under CSR) (Wilson, et al. PLOS Computational Biology, 2022 18 (3): e1009900). The degree of spatial clustering was defined as high (>median) or low (≤median). We also assessed the cell abundance as absent, low (≤1% of total cells), or high (>1% of total cells). This resulted in a 5 level categorial variable for the statistical analysis: absent, low abundance/low spatial clustering (LL), low abundance/high spatial clustering (LH), high abundance/low spatial clustering (HL), and high abundance/high spatial clustering (HH). Illustrative examples of TMA cores with high/low abundance and high/low spatial clustering are shown in FIG. 2. For cell types that were not present in >70% of cores, the following three level variable was instead used: Absent, PL (present/low spatial clustering) and PH (present/high spatial clustering). The weighted kappa statistic was used to compare classifications each core was given (HH, HL, LH, LL, and Absent) across the different cell marker combinations (Cohen, et al. Psychol Bull, 1968 70 (4): 213-20).

### Survival Analysis of Univariate Clustering

Cox proportional hazards models were used to estimate hazard ratios (HR) and 95% confidence intervals (CI) of the five- or three-level categories of abundance and spatial cluster with overall survival using the R package survival v3.3.1 (Therneau. A Package for Survival Analysis in R. 2022). Samples were restricted to those with invasive HGSOC with a survival time longer than 1 month. The models were adjusted for year of diagnosis (continuous), age at diagnosis (continuous), stage (½, ¾), and study (NHS, NHSII, and NECC). For the analysis of T cell subsets (e.g., CD3⁺CD4⁺Foxp3⁺ and CD3⁺CD8⁺), the models were further adjusted for the percentage of cells in the tumor compartment that were CD3⁺. Additionally, the models accounted for repeated measures (i.e., multiple cores per patient) by including a cluster effect in the Cox model. P-values lower than 0.01 were considered statistically significant. In cases where global test for association was significant, we explored individual levels in the model for their differences from the “Absent” group. This was also followed for the bivariate associations where individual levels were comped to “Present-Present-None”. Models were also fit to samples with HH and HL abundance/spatial context to determine differences. To determine whether the addition of the spatial context to the model significantly improved fit, LRT between abundance+spatial models and models with only abundance was performed.

### Survival Analysis of Bivariate Colocalization

Similar to the analysis approach for univariate clustering, Cox models were used for assessment of the association between the bivariate colocalization of two cell phenotypes and overall survival. Since CD3⁺ and CD3⁺CD8⁺ cells produced significant associations between the level of spatial clustering and survival, bivariate analysis examined the colocalization of CD3⁺CD8⁺with CD3⁺CD4⁺, CD3⁺CD4⁺Foxp3⁺, CD3⁺CD4⁺CD69⁺, and CD3⁺CD8⁺CD69⁺with CD3⁺CD4⁺ and CD3⁺CD4⁺CD69⁺. Colocalization of CD3⁺CD8⁺with CD3⁺CD4⁺can be interpreted as the clustering of CD3⁺CD4⁺around CD3⁺CD8⁺ and can only be computed when both marker combinations are present in the tissue sample. When both markers were present, the degree of spatial clustering was categorized as low or high based on the median. Thus, the bivariate analyses resulted in the following possible colocalization categories: AAN=absent/absent/no clustering assessed, APN=absent/present/no clustering assessed, PAN=present/absent/no clustering assessed, PPH=present/present/high degree of colocalization, and PPL=present/present/low degree of colocalization. Statistical modeling was conducted as described for univariate clustering using Cox proportional hazards models with all levels, PPH and PPL, as well to determine benefit of adding spatial information. P-values less than 0.01 were considered statistically significant.

### Spatial Contribution and Sensitivity Analysis

To test whether adding spatial clustering information improved model fit, a likelihood ratio test (LRT) was conducted to compare models with and without the inclusion of spatial clustering information. If the global test for association was significant, the individual-level significance for each group was compared to the “absent” reference group. Subset analysis involving only high-abundance samples was also performed to assess the difference between high and low spatial clustering in these samples. Sensitivity analyses were performed restricted to Non-Hispanic White women, as well as removing samples that were collected over 30 years before assays were completed (and thus may have been affected by reduced antigenicity).

### Results

### Univariate Degree of Spatial Clustering

Table 1 shows the women with HGSOC and mIF data that were included in this study. A total of 1244 TMA cores from 433 patients were identified (NECC=175, NHS=207, NHSII=51) with median age at diagnosis ranging from 53.7 years old (NHSII) to 67.1 years old (NHS). The CD3 marker was positive in more than 90% of the TMA cores with enough positive cells to calculate the degree of spatial clustering in 83.9% of sample cores, the highest percentage of all markers or combinations of markers (FIG. 9—Supplemental Table 2). The degree of spatial clustering requires at least two cells to enable estimation; therefore, spatial clustering analysis was not completed for cores with a single positive CD3⁺ cell. The proportion of cores positive for CTLs or recently activated CTLs was slightly lower than that of total TILs (75.6% and 60.5%, respectively, and 65.8% and 49.4%, respectively, with two or more cells for spatial clustering calculations). However, the abundance was lower for helper T cells (51.3% of samples with at least one positive cell and 38.8% having two or more positive cells), recently activated helper T cells (24.5% of cores having only a single positive cell and 14.9% of cores having two or more positive cells) and regulatory T cells (Tregs; 39.8% of cores with at least one positive cell and 27.4% cores with two or more). The 5-level abundance/spatial clustering categorization was used for all cell types except recently activated helper T cells, which used the 3-level abundance/spatial categories.

TABLE 1
Summary of patients by study restricted to include only those
who had tumors that were high grade serous with stages from
I-IV who lived for more than a month after surgery.

| Cohort | NECC | NHS | NHSII |
|---|---|---|---|
| Total Patients | 175 | 207 | 51 |
| Age at Diagnosis (years) | 59.12 | 67.08 | 53.71 |

Stage

| | | | |
|---|---|---|---|
| Stage I | 16 | 19 | 6 |
| Stage II | 17 | 12 | 6 |
| Stage III | 132 | 165 | 36 |
| Stage IV | 10 | 11 | 3 |

Vital Status

| | | | |
|---|---|---|---|
| Died | 131 | 186 | 28 |
| Alive | 44 | 21 | 23 |

Race

| | | | |
|---|---|---|---|
| White (Non-Hispanic) | 170 | 200 | 51 |
| White (Hispanic) | 2 | 3 | 0 |
| Black | 2 | 0 | 0 |
| Asian | 1 | 0 | 0 |
| American Indian | 0 | 1 | 0 |
| Median Follow-up (months) | 56.6 | 48.0 | 79.0 |

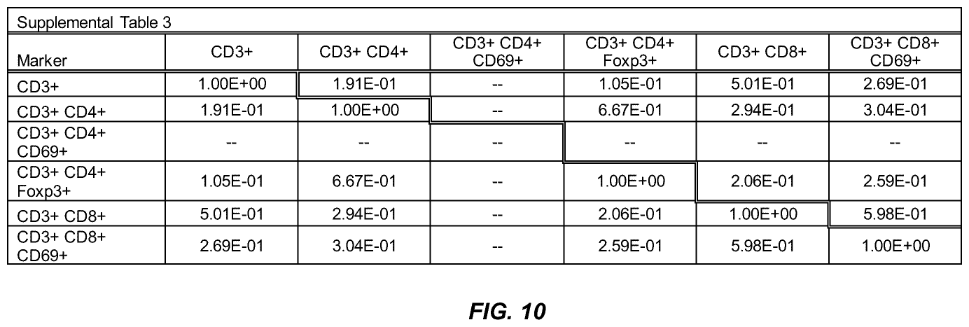

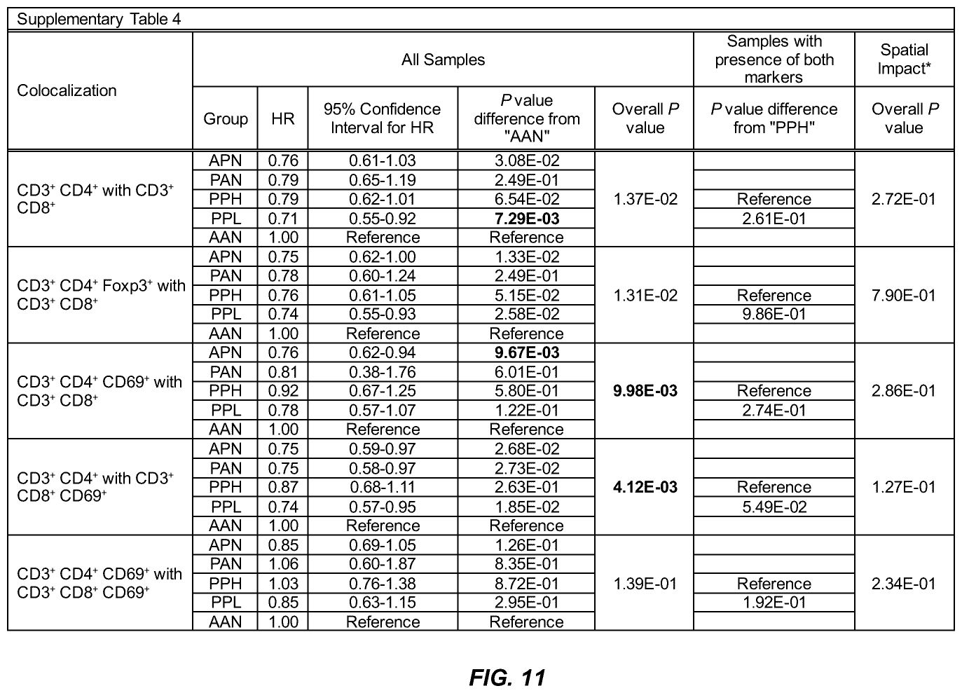

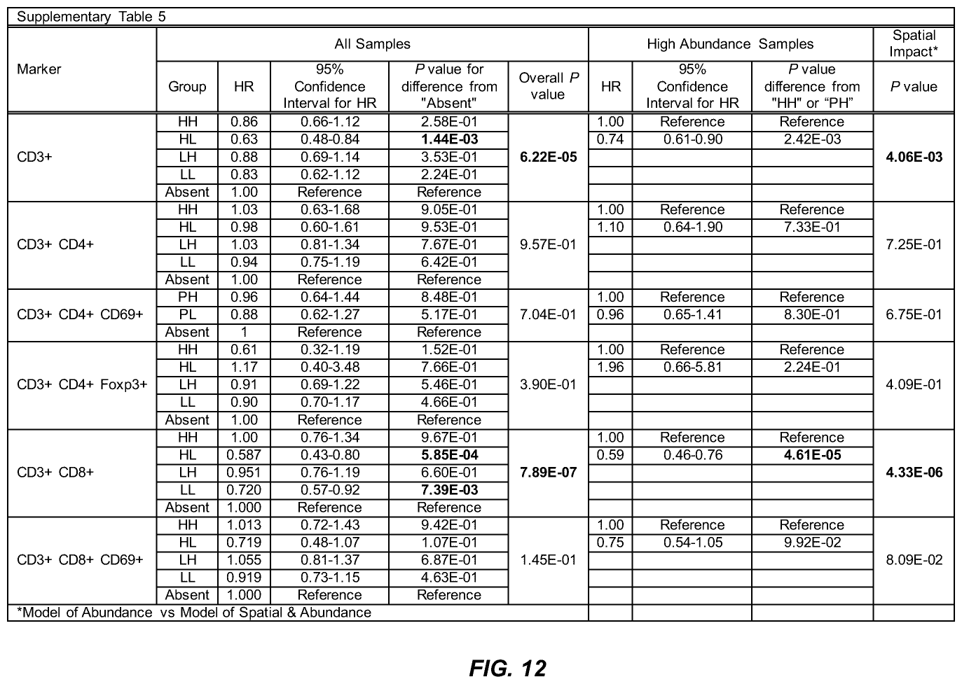

Helper T cells and Tregs had the highest concordance in the classification of abundance and spatial distribution (weighted kappa statistic [Kw]=0.67; FIG. 10—Supplemental Table 3). Derivative cell types (i.e., those that demonstrated additional differentiation) tended to show higher agreement than cell types with different differentiation pathways. For example, helper T cells and regulatory T cells (CD3⁺CD4⁺→CD3⁺CD4⁺FOXP3⁺) as well as CTLs and recently activated CTLs (CD3⁺CD8⁺→CD3⁺CD8⁺CD69⁺) had similar kappa statistics (Kw=0.60). Moderate concordance was observed between CD3+ T cells and CTLs (Kw=0.50). Conversely, helper and cytotoxic T cells showed weak agreement between the class assignments (Kw=0.29).

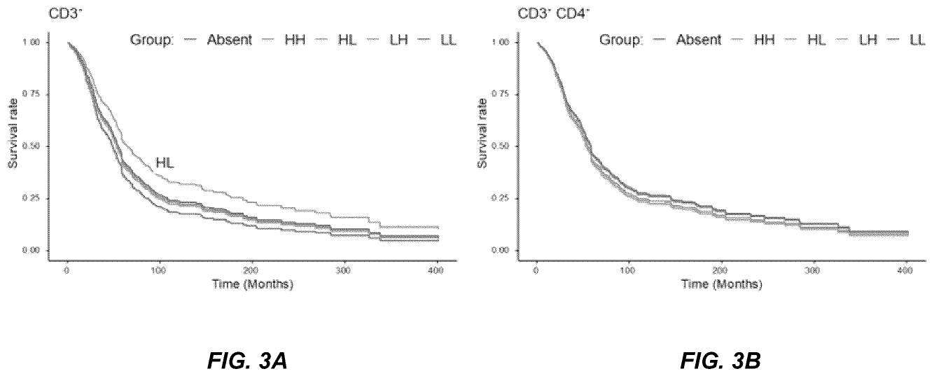

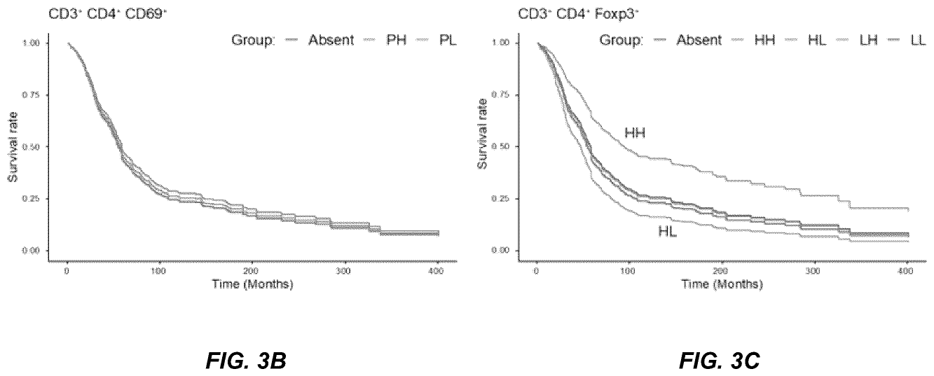

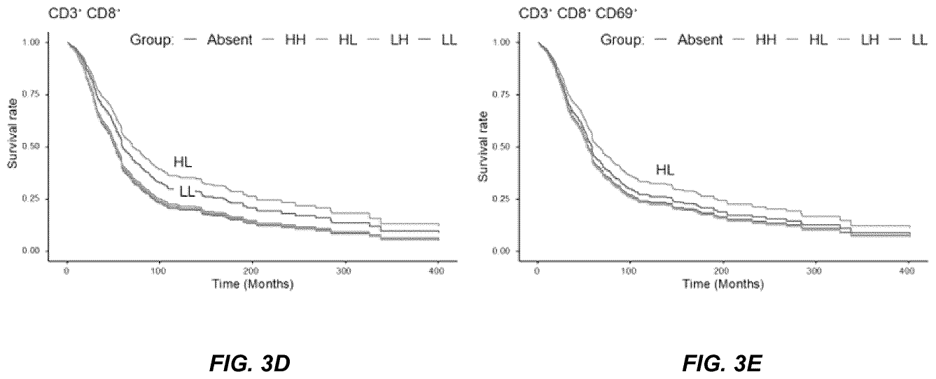

Models with the 5-levels of abundance/spatial contexture and clinical features showed significantly better fit than models with only clinical features for TILs (P=0.00001) and CTLs (P=0.000003), but not Tregs, recently activated helper T cells, and recently activated cytotoxic T cells (Table 2, FIG. 3). Patients with tumor samples with a high abundance and low degree of spatial clustering of TILs had significantly better overall survival than patients with tumor tissue samples with no TILs (HR=0.61, 95% CI 0.46-0.81, P=0.0005). This trend was also observed for CTLs (HR=0.59, 95% CI 0.44-0.80, P=0.0007). Other levels of abundance/spatial clustering did not show differing relationships with survival compared to the reference group (i.e., tumor absent of corresponding immune cell), except for low abundance/low spatial clustering of CTLs (HR=0.72, 95% CI 0.57-0.92, P=0.007). Tregs, helper T cells, recently activated helper T cells, and recently activated CTLs did not meet our multiple testing correction threshold (P<0.01), with a range of p-value from 0.02 for high abundance/high spatial clustering of Tregs to 0.98 for high abundance/low spatial clustering of helper T cells.

TABLE 2
Univariate Clustering.

| | | All Samples | | | |
|---|---|---|---|---|---|
| | | | 95% | P value for | |
| | | | Confidence | difference | |
| | | | Interval | from | Overall |
| Marker | Group | HR | for HR | “Absent” | P value |
| CD3+ | HH | 0.84 | 0.65-1.09 | 1.82E−01 | 1.08E−05 |
| | HL | 0.61 | 0.46-0.81 | 5.10E−04 | |
| | LH | 0.87 | 0.68-1.11 | 2.53E−01 | |
| | LL | 0.81 | 0.61-1.09 | 1.61E−01 | |
| | Absent | 1.00 | Reference | Reference | |
| CD3+ CD4+ | HH | 0.90 | 0.56-1.44 | 6.70E−01 | 8.51E−01 |
| | HL | 1.00 | 0.60-1.68 | 9.88E−01 | |
| | LH | 1.04 | 0.81-1.35 | 7.39E−01 | |
| | LL | 0.92 | 0.73-1.16 | 5.03E−01 | |
| | Absent | 1.00 | Reference | Reference | |
| CD3+ CD4+ | PH | 0.94 | 0.63-1.41 | 7.70E−01 | 6.25E−01 |
| CD69+ | PL | 0.88 | 0.62-1.24 | 4.55E−01 | |
| | Absent | 1.00 | Reference | Reference | |
| CD3+ CD4+ | HH | 0.51 | 0.29-0.90 | 1.94E−02 | 7.87E−02 |
| Foxp3+ | HL | 1.29 | 0.45-3.73 | 6.33E−01 | |
| | LH | 0.93 | 0.69-1.23 | 5.96E−01 | |
| | LL | 0.91 | 0.70-1.17 | 4.46E−01 | |
| | Absent | 1.00 | Reference | Reference | |
| CD3+ CD8+ | HH | 0.97 | 0.72-1.30 | 8.28E−01 | 2.85E−06 |
| | HL | 0.59 | 0.44-0.80 | 6.83E−04 | |
| | LH | 0.93 | 0.75-1.16 | 5.40E−01 | |
| | LL | 0.72 | 0.57-0.92 | 7.36E−03 | |
| | Absent | 1.00 | Reference | Reference | |
| CD3+ CD8+ | HH | 1.03 | 0.72-1.47 | 8.93E−01 | 1.98E−01 |
| CD69+ | HL | 0.74 | 0.49-1.11 | 1.48E−01 | |
| | LH | 1.03 | 0.80-1.33 | 8.01E−01 | |
| | LL | 0.91 | 0.73-1.13 | 3.76E−01 | |
| | Absent | 1.00 | Reference | Reference | |

| | | High Abundance Samples | | | |
|---|---|---|---|---|---|
| | | | 95% | P value | |
| | | | Confidence | difference | Spatial |
| | | | Interval | from | Impact* |
| Marker | Group | HR | for HR | “HH” or “PH” | P value |
| CD3+ | HH | 1.00 | Reference | Reference | 2.34E−03 |
| | HL | 0.73 | 0.60-0.89 | 2.09E−03 | |
| | LH | | | | |
| | LL | | | | |
| | Absent | | | | |
| CD3+ CD4+ | HH | 1.00 | Reference | Reference | 5.40E−01 |
| | HL | 1.20 | 0.69-2.11 | 5.21E−01 | |
| | LH | | | | |
| | LL | | | | |
| | Absent | | | | |
| CD3+ CD4+ | PH | 1.00 | Reference | Reference | 6.95E−01 |
| CD69+ | PL | 0.98 | 0.67-1.42 | 9.01E−01 | |
| | Absent | | | | |
| CD3+ CD4+ | HH | 1.00 | Reference | Reference | 1.32E−01 |
| Foxp3+ | HL | 1.96 | 0.66-5.81 | 2.24E−01 | |
| | LH | | | | |
| | LL | | | | |
| | Absent | | | | |
| CD3+ CD8+ | HH | 1.00 | Reference | Reference | 3.05E−05 |
| | HL | 0.59 | 0.45-0.78 | 2.10E−04 | |
| | LH | | | | |
| | LL | | | | |
| | Absent | | | | |
| CD3+ CD8+ | HH | 1.00 | Reference | Reference | 9.93E−02 |
| CD69+ | HL | 0.75 | 0.53-1.08 | 1.21E−01 | |
| | LH | | | | |
| | LL | | | | |
| | Absent | | | | |

Survival analysis results using cellular abundance (1% threshold for low vs high, unless more than 70% cores have the marker missing, present/absent is used) and Ripley's K degree of clustering (median as threshold for low vs high). Models were adjusted for age at diagnosis, high/low stage, and patient cohort and CD3 abundance for T cell subsets. Absent = no immune marker present in tumor core; LL = low abundance/low spatial clustering, LH = low abundance/high spatial clustering; HL = high abundance/low spatial clustering; HH = high abundance/high spatial clustering, PL = present marker/low spatial clustering, PH = present marker/high spatial clustering. Bolded values indicate results that are statistically significant with p-value < 0.01.

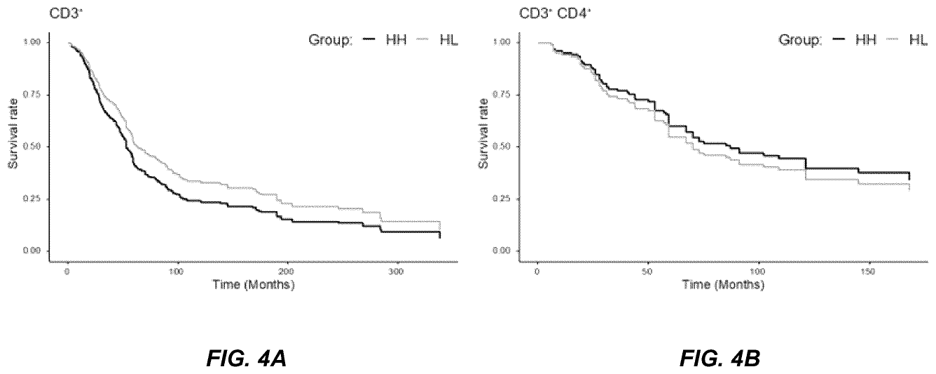

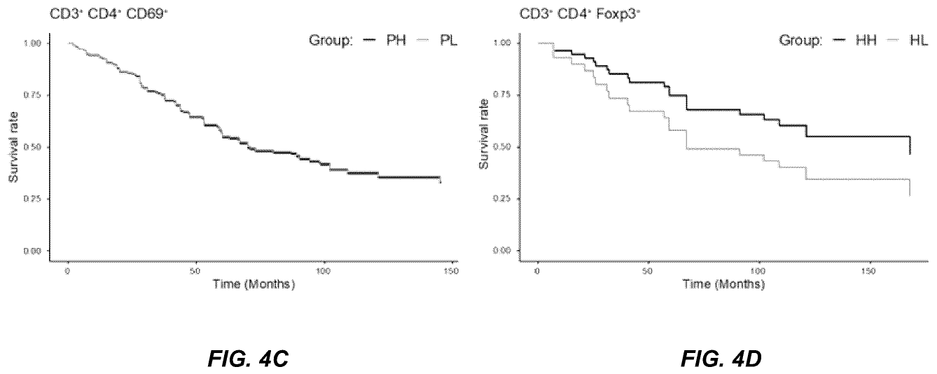

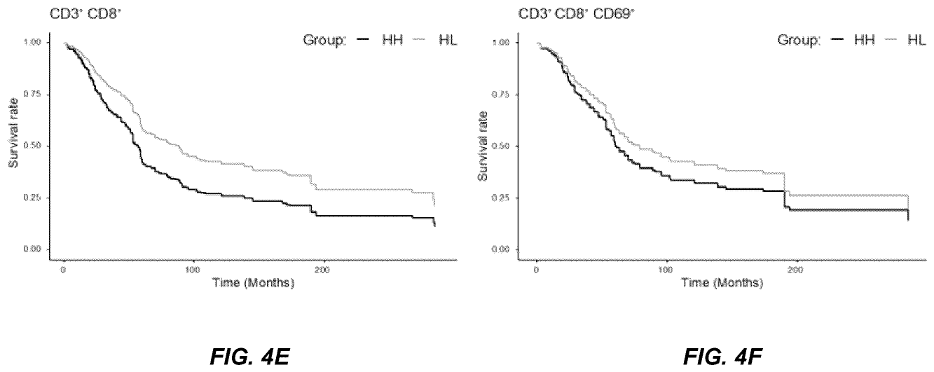

When restricted to TMA cores that had more than 1% abundance for the specific T cell subtype (i.e., high abundance), models for TILs and CTLs were significant and low spatial clustering had significantly better overall survival compared to high spatial clustering (TILs P=0.002, CTLs P=0.0002, Table 2, FIG. 4). Models for Tregs, helper T cells, recently activated helper T cells, and recently activated CTLs didn't show any association between abundance/spatial information and survival (P>0.12). Critically, the LRT comparing the fit of the model with levels of abundance/degree of spatial clustering to the model of abundance only for TILs and CTLs showed that the addition of spatial information to the Cox model was significantly better than the Cox model without spatial information (P=0.002 and P=0.00003, respectively).

### Bivariate Degree of Spatial Clustering

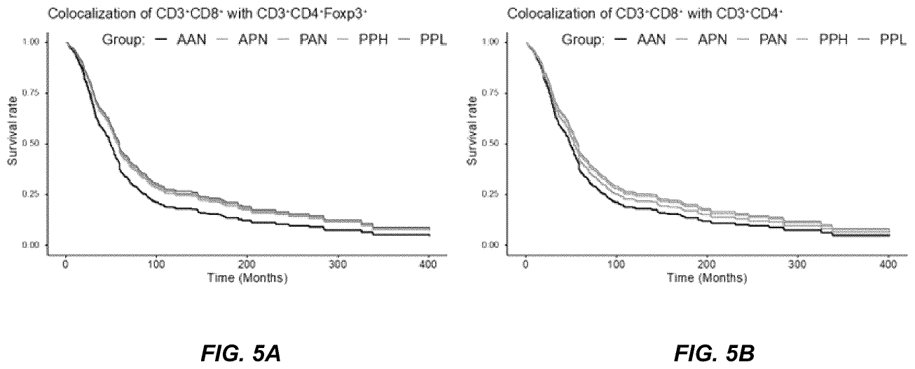

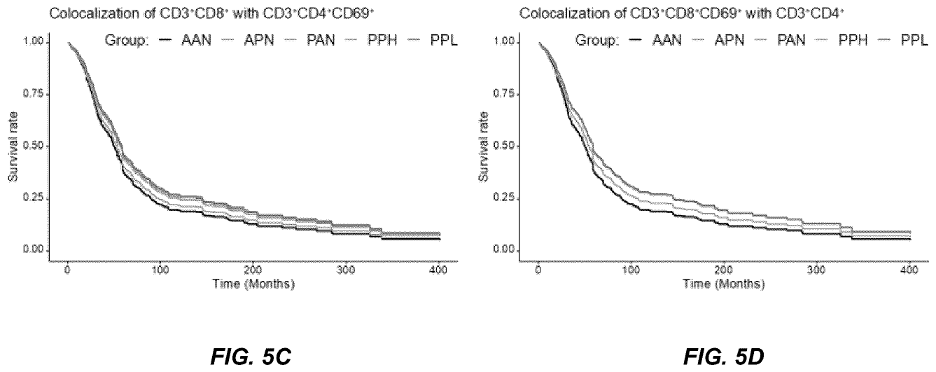

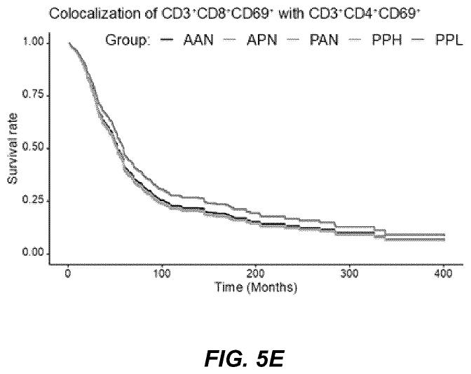

Colocalization of immune cells was tested for total or recently activated CTLs with Tregs, helper T cells, and recently activated helper T cells, as CTLs had significant association from the univariate clustering analyses. There was a significant association between overall survival and the five categories of abundance and spatial colocalization of CTLs with Tregs (P=0.012), CTLs with helper T cells (P=0.01), CTLs with recently activated helper T cells (P=0.01), and recently activated CTLs with helper T cells (P=0.005) (FIG. 5, Table 3). In comparisons of the categories of abundance and colocalization to the group with neither marker present (AAN, “reference” group), PPL (both markers present and low level of colocalization) for CTLs with helper T cells and PAN (CTLs present and helper T cells absent) of CTLs with recently activated helper T cells showed significant difference (HR=0.70, 95% CI: 0.54-0.91, P=0.006 and HR=0.76, 95% CI: 0.62-0.94, P=0.01, respectively). However, the Cox models with colocalization information (in addition to abundance) were not statistically better than the Cox model with only abundance information (P=0.25 and P=0.3, respectively).

TABLE 3
Bivariate Clustering.

| | | | | | | Samples | |
|---|---|---|---|---|---|---|---|
| | | | | | | with | |
| | | | | | | presence | |
| | | | | | | of both | |
| | All Samples | | | | | markers | Spatial |
| | | | 95% | p-value | | P value | Impact* |
| | | | Confidence | difference | Overall | difference | Overall |
| | | | Interval for | from | P | from | P |
| Colocalization | Group | HR | HR | “AAN” | value | “PPH” | value |
| CD3+ CD8+ | APN | 0.78 | 0.60-1.24 | 2.49E−01 | 1.18E−02 | | 5.77E−01 |
| with CD3+ | PAN | 0.75 | 0.62-1.00 | 1.33E−02 | | | |
| CD4+ | PPH | 0.78 | 0.61-1.06 | 6.81E−02 | | Reference | |
| Foxp3+ | PPL | 0.73 | 0.54-0.92 | 1.93E−02 | | 7.28E−01 | |
| | AAN | 1.00 | Reference | 1.00 | | | |
| CD3+ CD8+ | APN | 0.86 | 0.65-1.19 | 3.36E−01 | 1.04E−02 | | 2.52E−01 |
| with CD3+ | PAN | 0.76 | 0.61-1.03 | 2.88E−02 | | | |
| CD4+ | PPH | 0.79 | 0.62-1.02 | 5.79E−02 | | Reference | |
| | PPL | 0.70 | 0.54-0.91 | 5.93E−03 | | 2.75E−01 | |
| | AAN | 1.00 | Reference | 1.00 | | | |
| CD3+ CD8+ | APN | 0.81 | 0.38-1.76 | 6.00E−01 | 1.03E−02 | | 3.00E−01 |
| with CD3+ | PAN | 0.76 | 0.62-0.94 | 9.62E−03 | | | |
| CD4+ | PPH | 0.91 | 0.66-1.26 | 5.76E−01 | | Reference | |
| CD69+ | PPL | 0.78 | 0.57-1.07 | 1.22E−01 | | 2.56E−01 | |
| | AAN | 1.00 | Reference | 1.00 | | | |
| CD3+ CD8+ | APN | 0.75 | 0.58-0.97 | 2.71E−02 | 4.96E−03 | | 1.68E−01 |
| CD69+ with | PAN | 0.75 | 0.59-0.97 | 2.68E−02 | | | |
| CD3+ | PPH | 0.86 | 0.67-1.10 | 2.34E−01 | | Reference | |
| CD4+ | PPL | 0.74 | 0.57-0.96 | 2.16E−02 | | 8.16E−02 | |
| | AAN | 1.00 | Reference | 1.00 | | | |
| CD3+ CD8+ | APN | 1.06 | 0.60-1.87 | 8.36E−01 | 1.23E−01 | | 1.89E−01 |
| CD69+ with | PAN | 0.85 | 0.69-1.05 | 1.25E−01 | | | |
| CD3+ CD4+ | PPH | 1.03 | 0.78-1.38 | 8.25E−01 | | Reference | |
| CD69+ | PPL | 0.84 | 0.62-1.14 | 2.69E−01 | | 1.09E−01 | |
| | AAN | 1.00 | Reference | 1.00 | | | |

Survival analysis results from colocalization of CD3+CD8+ or CD3+CD8+CD69+ cells with CD3+CD4+Foxp3+, CD3+CD4++, or CD3+CD4+CD69+ using bivariate Ripley's K (median as threshold for low (L) vs high (H)) with cell abundance (present (P) vs absent (A)). Models were adjusted for age at diagnosis, high/low stage, and patient cohort and CD3 abundance for T cell subsets. APN = absent of CD3+CD8+ or CD3+CD8+CD69+ (A), present of other phenotype (CD3+CD4+, CD3+CD4+Foxp3+, or CD3+CD4+CD69+) (P), and no spatial clustering computed (N); PAN = presence of CD3+CD8+ or CD3+CD8+CD69+ (P), absence of other phenotype (CD3+CD4+, CD3+CD4+Foxp3+ , or CD3+CD4+CD69+) (A), and no spatial clustering computed (N); PPH = presence of both phenotypes and high level of colocalization; PPL = presence of both phenotypes and low level of colocalization; AAN = absence of both phenotypes and no level of colocalization computed. Bolded values indicate results that are statistically significant with p-value <0.01.

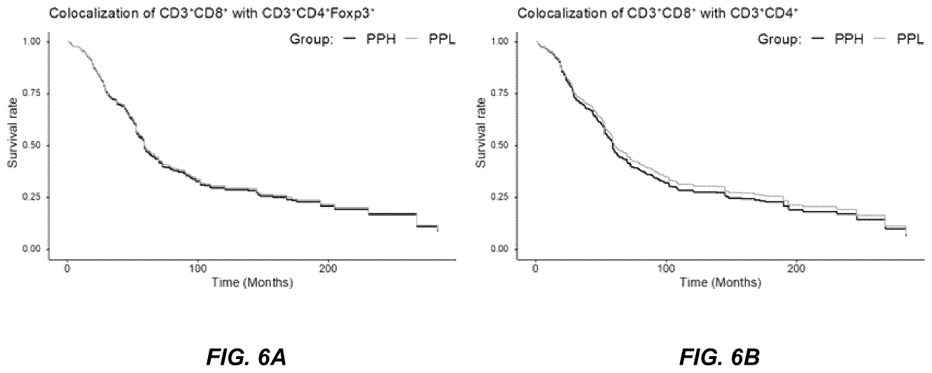

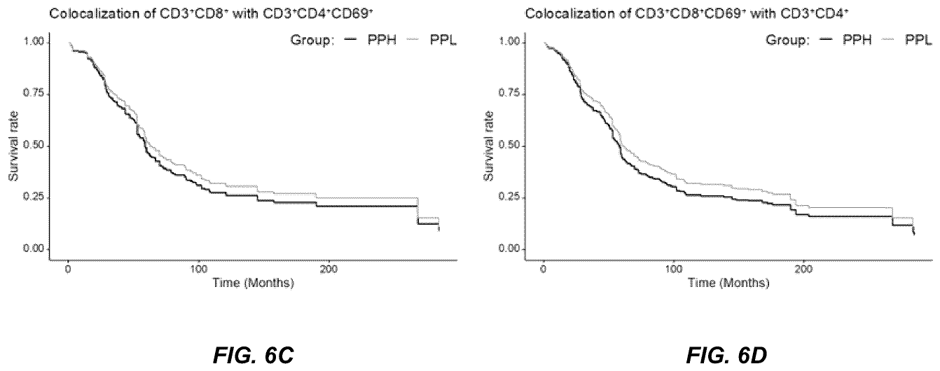

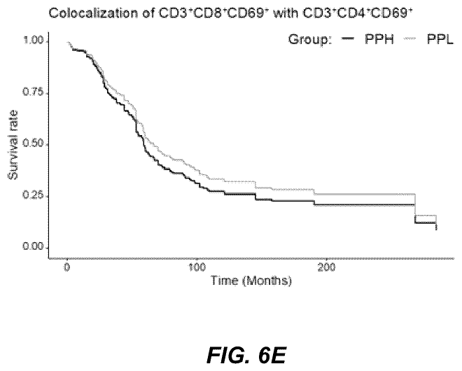

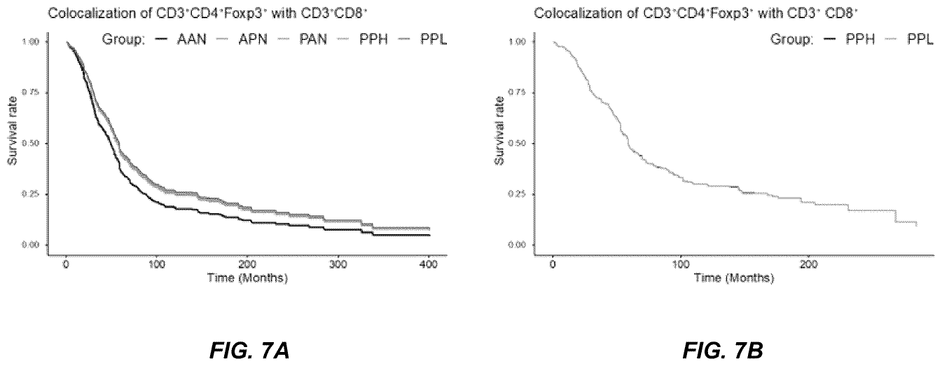

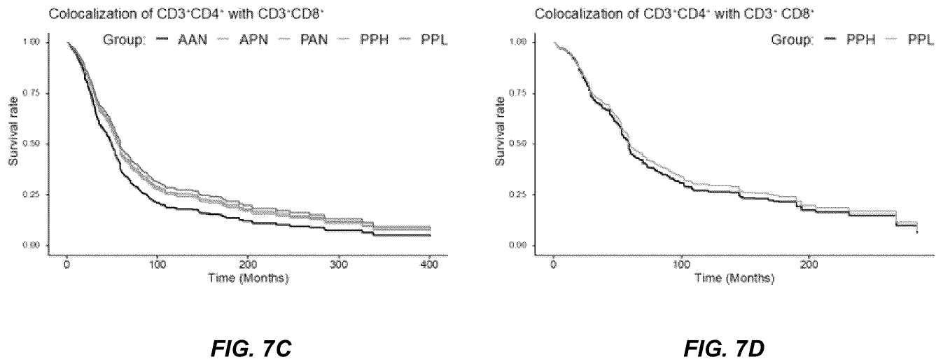

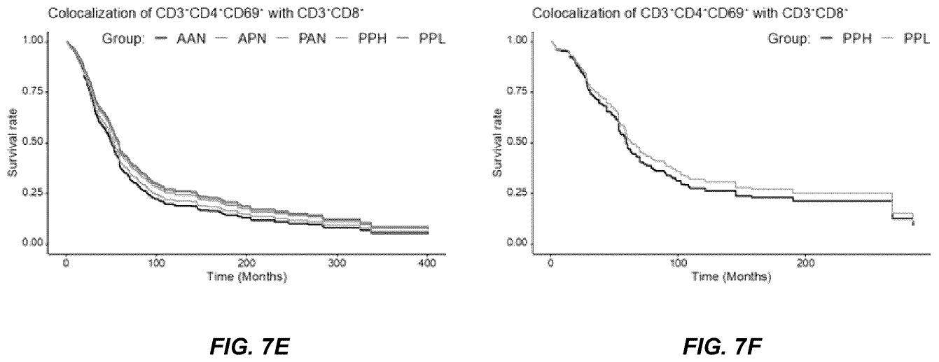

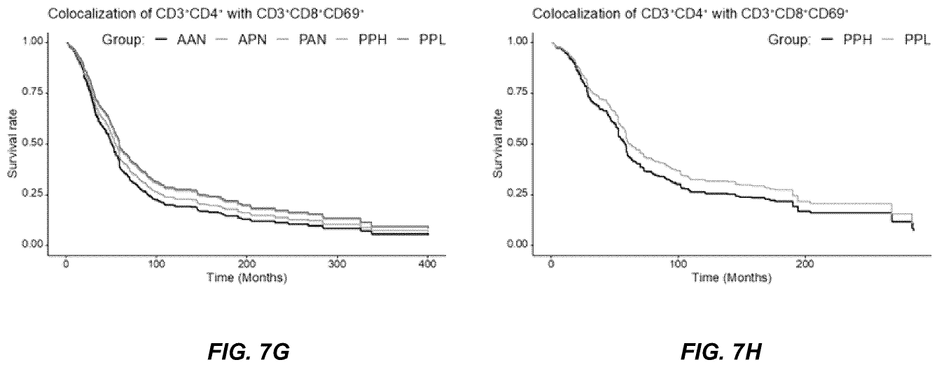

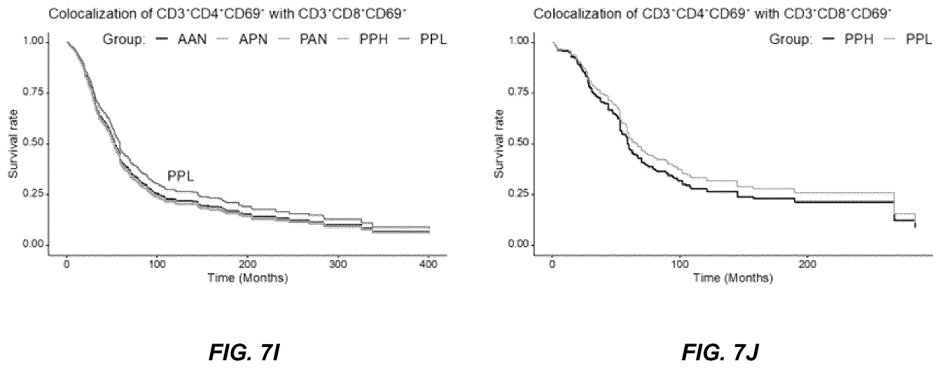

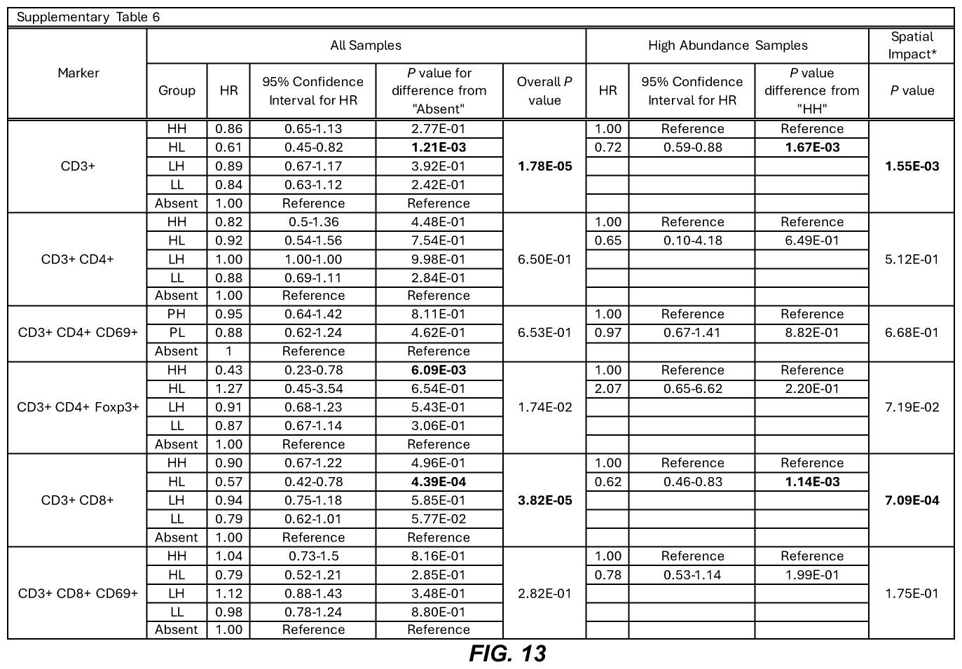

When restricted to cores that have both cell types of interest present, there was no significant difference in the associations with survival based on the level of colocalization for any of the marker combinations assessed (e.g., low vs high colocalization) (FIG. 6, Table 3). The most significant difference in survival between high and low colocalization was observed for recently activated CTLs and helper T cells (P=0.08), whereby a low level of colocalization was found to be associated with better survival. Inverting the colocalization cell type assignments for computing Ripley's K, showed a similar suggestive association of survival with the colocalization of helper T cells with recently activated CTLs (P=0.054; FIG. 13—Supplemental Table 4).

### Sensitivity Analysis

When restricted to non-Hispanic white patients, no substantive differences in the main study findings were observed, with high abundance/low spatial clustering of CTLs maintaining strong association with overall survival (FIG. 13—Supplemental Table 5). Similar results were observed when the analysis was restricted to tumor samples collected within 30 years of the assay (FIG. 14—Supplemental Table 6). However, even though low abundance/low degree of spatial clustering (LL) of CTLs association with survival was no longer different from the absent group (HR=0.79, 95% CI: 0.62-1.01), the model for CTLs still showed that including both abundance and spatial contexture improved model fit when compared to a model with only abundance (P=0.0007).

### DISCUSSION

HGSOC is a lethal disease, and little is known about how the spatial architecture of the TIME is related to survival. It was previously shown that high abundance and a low degree of spatial clustering of TILs and CTLs in the tumor compartment was associated with better overall survival among non-Hispanic Black women with HGSOC, and that the addition of the spatial contexture to the classic cellular abundance in the samples significantly improved the statistical model fit over a model using only cellular abundance (Wilson, et al. PLOS Computational Biology, 2022 18 (3): e1009900). In this study, these prior findings were replicated in a large study to show predominantly non-Hispanic White women with HGSOC tumors containing high abundance and low degree of spatial clustering of tumor-infiltrating lymphocytes and cytotoxic T cells had the best overall survival. Furthermore, considering both the degree of spatial clustering and cell abundance significantly improved model fit compared to the standard use of abundance alone as a prognostic marker. Additionally, low colocalization of CTLs with either helper T cells or recently activated helper T cells (PPL) was related to a modest survival benefit compared to the presence of either cell type (PAN or APN), although the model with colocalization information was not better than having each cells abundance in the model. In general, the spatial architecture of other T cell subsets did not significantly predict overall survival following a HGSOC diagnosis.

The presence of TILs and CTLs within the HGSOC tumor compartment has been well characterized and abundance is strongly associated with longer overall survival (Zhang, et al. New England Journal of Medicine, 2003 348 (3): 203-213; Sato, et al. Proc Natl Acad Sci USA, 2005 102 (51): 18538-18543). TILs can recognize cancer cells, over-express self-antigens, and secrete cytokines that aid in the recruitment of other TILs, such as CTLs (Kaech, et al. Nat Rev Immunol, 2012 12 (11): 749-6; Drakes, et al. Cancers (Basel), 2018. 10 (9)). CTLs release molecules (e.g., cytokines) that either directly kill tumor cells or recruit more immune cells to the TIME. Overall, experimental evidence and population studies support that tumor infiltration of TILs and CTLs are related to improved patient survival (Drakes, et al. Cancers (Basel), 2018. 10(9)). A possible biological explanation for our observations regarding the spatial contexture is that tumors presenting a diffuse pattern of CTLs and TILs may represent a broader anti-tumor immune response and do so across the whole tumor. Notably, recent studies have shown that stem-like CTLs, which have high proliferative and differentiation potential, associated with an improved response to immunotherapy (Tooley, et al. Trends Cancer, 2022 8 (8): 642-654; Stoltzfus, et al. Front Immunol, 202112:726492). Another hypothesis is that spatial contexture may be a surrogate measure for structural differences in the overall tumor microenvironment driven either by the immune response or by tumor development. For example, stem-like CTLs appear to generally remain at the tumor border and differentiated effector CD8+ T cells tend to be more proximal to the antigen-producing cells; this spatial positioning (or restriction) is in part due to chemokine and cytokine secretion in the tumor microenvironment (Tooley, et al. Trends Cancer, 2022 8 (8): 642-654). Although this study did not measure all markers representing the different T cell subtypes, low spatial clustering could represent this type of T cell distribution capturing both tumor border effects and intra-tumoral T cells. Further research should evaluate how other subsets of T cells are distributed within the tumor and stroma, as well as, with respect to tumor vasculature.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a subject with high grade serous ovarian cancer to immunotherapy, comprising
a) conducting multiplex immunofluorescence (mIF) microscopy on a tumor sample from the subject for the abundance and spatial clustering of cells expressing CD3, CD4, CD8, and CD69 compared to a control;
b) detecting high abundance and low spatial cluster of CD3⁺CD4⁻CD8⁻ cells, or detecting high or low abundance and low spatial cluster of CD3⁺CD4⁻CD8⁺CD69⁻ cells; and
c) treating the subject with immunotherapy

2. The method of claim 1, wherein the immunotherapy is a T cell immunotherapy.

3. The method of claim 2, wherein the T cell immunotherapy comprises a chimeric antigen receptor (CAR) T-cell therapy or tumor-infiltrating lymphocyte (TIL) therapy.

4. The method of claim 1, wherein the immunotherapy comprises a checkpoint inhibitor.

5. The method of claim 4, wherein the checkpoint inhibitor comprises an anti-PD-1 antibody, anti-PD-L1 antibody, anti-CTLA-4 antibody, or a combination thereof.

6. The method of claim 1, wherein the solid tumor comprises a sarcoma, carcinoma, or lymphoma.
